(19) United States
(12) Patent Application Publication
(10) Pub. No.: US 2015/0258139 A1
(43) Pub. Date: Sep. 17, 2015

(54) METHODS FOR TREATMENT OF CANCER USING LIPOPLATIN

(71) Applicants: Teni Boulikas, Alimos, Attiki (GR); George Stathopoulos, Athens, Attiki (GR)

(72) Inventors: Teni Boulikas, Alimos, Attiki (GR); George Stathopoulos, Athens, Attiki (GR)

(73) Assignee: REGULON, INC., Palo Alto, CA (US)

(21) Appl. No.: 14/390,352

(22) PCT Filed: Aug. 13, 2012
(86) PCT No.: PCT/US2012/050626

§ 371 Date: May 7, 2015

(51) Int. Cl. A61K 33/24; A61N 5/10; A61K 31/495; A61K 9/127; A61K 9/00; A61K 45/06

(52) U.S. Cl. CPC A61K 33/24; A61K 9/0019; A61K 45/06; A61K 31/495; A61K 9/127; A61N 5/1084; A61N 2005/1098

(57) ABSTRACT

Applicant provides herein a method for inhibiting the growth of a solid tumor or treating cancer in a patient comprising, or alternatively consisting essentially of, or yet further consisting of administering to the patient an effective amount of Lipoplatin monotherapy in a first dose and a second dose, thereby inhibiting the growth of the solid tumor or treating cancer in the patient.

### BACKGROUND

The present invention relates generally to the field of solid tumors that are responsive to platinum therapy.

Cisplatin has been in use for over 30 years and has been demonstrated to be an effective agent against a number of malignancies, including lung, ovarian, head and neck, gynecological, testicular and urothelial cancers (2-10).

Although cisplatin is one of the most significant and effective anticancer agents, its toxicity is often an inhibiting factor preventing the continuation of treatment courses. The main side effect is renal toxicity (renal failure). Other adverse reactions have included nausea and vomiting, asthenia and neurotoxicity (11-14).

Over the last 15-20 years, there has been an extensive effort to produce other agents as a substitute for cisplatin. The main substitutive agent was the CDDP analogue, carboplatin. Moreover, in certain malignancies other new agents, including taxanes (paclitaxel, docetaxel) and gemcitabine and vinorelbine, have been tested. Renal toxicity was avoided with the use of these agents, but other side effects, including myelotoxicity, were observed. However, none of these agents were more effective when compared with cisplatin (15-21).

Thus, a need exists for an effective treatment which is relatively non-toxic. This invention satisfies this need and provides related advantages as well.

### SUMMARY

Applicant herein reports a study that compared Lipoplatin therapy with conventional cisplatin therapy with respect to toxicity and effectiveness. As a result, Applicant provides a method for inhibiting the growth of a solid tumor or treating cancer in a patient comprising, or alternatively consisting essentially of, or yet further consisting of administering to the patient an effective amount of Lipoplatin monotherapy in a first dose and a second dose, thereby inhibiting the growth of the solid tumor or treating cancer in the patient, with minimal toxicity.

This disclosure also provides a method for inhibiting the growth of a solid tumor or treating cancer in a patient comprising, or alternatively consisting essentially of, or yet further consisting of, administering a first dose of Lipoplatin monotherapy to the patient, wherein the first dose comprises about 200 mg/m2 and a second dose to the patient, of about 200 mg/m2 of Lipoplatin monotherapy about 24 hours after administration of the first dose, thereby inhibiting the growth of the tumor or treating the patient.

In another aspect, a method is provided for inhibiting the growth of a brain tumor or treating a brain tumor in a subject, comprising intra-arterial administration of an effective amount of Lipoplatin to the subject, thereby inhibiting the growth of the brain tumor or treating the brain tumor in the subject.

A pharmaceutical Lipoplatin composition is provided that comprises, or alternatively consists essentially of, or yet further consists of, an effective amount of Lipoplatin to provide a dose of from about 100 mg/m2 to about 300 mg/m2 in a pharmaceutically acceptable carrier. The composition can further contain an effective amount of a drug that enhances transport of the Lipoplatin across the blood brain barrier.

A kit is also provided by Applicant, that provides the compositions as disclosed herein and optionally, instructions for performing the methods of this disclosure.

### BRIEF DESCRIPTION OF THE FIGURES

FIG. 1 is a diagram of the chronological sequence for in vivo experimentations.

FIG. 2 shows uptake of the studied Platinum drugs, 24 hours after administration. Nucleus and cytoplasm are from the tumor sections. Tumor section was measured (not an addition of nucleus and cytoplasm). The term Contra lat refers to the healthy contra lateral hemisphere of the brain that does not contain the tumor. IV=intra-veinous, IA=intra-arterial, BBBD=blood—brain barrier disruption.

FIGS. 3A through F are Kaplan-Meier survival graphs for F98 glioma bearing rats. A) IV platinum alone (dashed lines) or combination with radiation (full lines). B) IA platinum alone (dashed lines) or combination with radiation (full lines). C) BBBD platinum alone (dashed lines) or combination with radiation (full lines). D) Carboplatin and Lipoplatin™ by IV, IA and BBBD. E) Oxaliplatin compared to its liposomal formulation, Lipoxal™, by IV, IA and BBBD. F) Cisplatin compared to its liposomal formulation, Lipoplatin™, by IV and IA. GK=Gamma Knife (15 Gy to the tumor volume plus a margin of 2 mm.)

### DETAILED DESCRIPTION

Throughout this application, the text refers to various embodiments of the present compositions and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

Also throughout this disclosure, various publications, patents and published patent specifications are referenced by an identifying citation or an Arabic number, the complete bibliographic citation for which is found immediately preceding the claims. The disclosures of these publications, patents and published patent specifications are hereby incorporated by reference into the present disclosure in their entirety to more fully describe the state of the art to which this invention pertains.

### DEFINITIONS

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients. Embodiments defined by each of these transition terms are within the scope of this invention.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above.

All numerical designations, e.g., pH, temperature, time, concentration, and molecular weight, including ranges, are approximations which are varied (+) or (−) by increments of 0.1 or 1.0 as is appropriate. It is to be understood, although not always explicitly stated that all numerical designations are preceded by the term “about” which includes a standard deviation of about 15%, or alternatively about 10% or alternatively about 5%. It also is to be understood, although not always explicitly stated, that the reagents described herein are merely exemplary and that equivalents of such are known in the art. An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. Studies in animal models generally may be used for guidance regarding effective dosages for treatment of diseases. In general, one will desire to administer an amount of the compound that is effective to achieve a serum level commensurate with the concentrations found to be effective in vitro. Thus, where a compound is found to demonstrate in vitro activity, for example as noted in the Tables discussed below one can extrapolate to an effective dosage for administration in vivo. These considerations, as well as effective formulations and administration procedures are well known in the art and are described in standard textbooks. Consistent with this definition and as used herein, the term “therapeutically effective amount” is an amount sufficient to treat a specified disorder or disease or alternatively to obtain a pharmacological response treating a glioblastoma.

As used herein, “treating” or “treatment” of a disease in a patient refers to (1) preventing the symptoms or disease from occurring in an animal that is predisposed or does not yet display symptoms of the disease; (2) inhibiting the disease or arresting its development; or (3) ameliorating or causing regression of the disease or the symptoms of the disease.

As understood in the art, “treatment” is an approach for obtaining beneficial or desired results, including clinical results. For the purposes of this invention, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Preferred are compounds that are potent and can be administered locally at very low doses, thus minimizing systemic adverse effects.

As used herein, “surgery” or “surgical resection” refers to surgical removal of a tumor of concern.

“Tumor Recurrence” as used herein and as defined by the National Cancer Institute is cancer that has recurred (come back), usually after a period of time during which the cancer could not be detected. The cancer may come back to the same place as the original (primary) tumor or to another place in the body. It is also called recurrent cancer.

“Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up.

“Disease free survival” indicates the length of time after treatment of a cancer or tumor, such as surgery, during which a patient survives with no signs of the cancer or tumor.

“Overall Survival” (OS) intends a prolongation in life expectancy as compared to naïve or untreated individuals or patients.

“Progressive Disease” (PD) intents a disease that is progressing or worsening. For example, with lung cancer, progressive disease can be a 20% growth in the size of the tumor or spread of the tumor since the beginning of treatment.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

“Monotherapy” as used herein refers to a therapy which is administered by itself. The term “determine” or “determining” is to associate or affiliate a patient closely to a group or population of patients who likely experience the same or a similar clinical response.

As used herein, the terms “Stage I cancer,” “Stage II cancer,” “Stage III cancer,” and “Stage IV” refer to the TNM staging classification for cancer. Stage I cancer typically identifies that the primary tumor is limited to the organ of origin. Stage II intends that the primary tumor has spread into surrounding tissue and lymph nodes immediately draining the area of the tumor. Stage III intends that the primary tumor is large, with fixation to deeper structures. Stage IV intends that the primary tumor is large, with fixation to deeper structures. See pages 20 and 21, CANCER BIOLOGY, 2^{nd} Ed., Oxford University Press (1987).

“Triple negative breast cancer” intends tumor that was tested for the expression of the markers: estrogen receptor (ER), the progesterone receptor (PR) and herceptin (HER2/neu), and is negative for all three markers.

Lipoplatin™ is a therapeutic composition and its method of making are described in U.S. Pat. Nos. 7,393,478 and 6,511,676, each incorporated by reference herein. The composition is described as a cisplatin micelle containing cisplatin in its aqua form, and obtainable by a method comprising, or alternatively consisting essentially of, or yet further consisting of: a) combining a suitable buffer solution, cisplatin with an effective amount of at least a 30% ethanol solution to form a cisplatin/ethanol solution; and b) combining the solution with a negatively charged phosphatidyl glycerol lipid derivative wherein the molar ratio between cisplatin and the lipid derivative is 1:1 to 1:2, thereby producing a cisplatin mixture in its aqua form in micelles. In one aspect, the ciplatin micelles are obtainable by a method that comprises, or alternatively consists essentially of, or yet further consists of: a) combining a suitable buffer solution, cisplatin with an effective amount of at least 30% ethanol solution to form a cisplatin/ethanol solution; and b) combining the cisplatin/ethanol solution with a negatively charged phosphatidyl glycerol lipid derivative wherein the molar ratio between cisplatin and the lipid derivative is 1:1 to 1:2, thereby producing a cisplatin mixture in its aqua form in micelles. In one aspect, the phosphatidyl glycerol lipid derivative is selected from the group consisting of dipalmitoyl phosphatidyl glycerol (DPPG), dimyristoyl phosphatidyl glycerol (DMPG), dicaproyl phosphatidyl glycerol (DCPG), distearoyl phosphatidyl glycerol (DSPG) and dioleyl phosphatidyl glycerol (DOPG). In another aspect, the molar ratio is 1:1. In a yet further aspect, the method to produce Lipoplatin further comprises, or alternatively consists essentially of, or yet further consists of combining an effective amount of a free fusogenic peptide, a fusogenic peptide-lipid conjugate or a fusogenic peptide-PEG-HSPC conjugate to the mixture of step a) where the fusogenic peptide is derivatized with a stretch of 1-6 negatively-charged amino acids at the N or C-terminus and thus, able to bind electrostatically to the cisplatin mixture in its aqua form. In one aspect, the free fusogenic peptide or fusogenic peptide lipid conjugate comprises, or alternatively consists essentially of, or yet further consists of, DOPE or DOPE/cationic lipid.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present invention is also useful for veterinary treatment of companion mammals, exotic animals and domesticated animals, including mammals, rodents, and the like.

The term administration shall include without limitation, administration by ocular, oral, intra-arterial, parenteral (e.g., intramuscular, intraperitoneal, inhalation, transdermal intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, ocular etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

A “pathological cell” is one that is pertaining to or arising from disease. Pathological cells can be hyperproliferative. A “hyperproliferative cell” means cells or tissue are dividing and growing at a rate greater than that when the cell or tissue is in a normal or healthy state. Examples of such include, but are not limited to cancer cells.

Hyperproliferative cells also include de-differentiated, immortalized, neoplastic, malignant, metastatic, and cancer cells such as sarcoma cells, leukemia cells, carcinoma cells, or adenocarcinoma cells. Specified cancers include, but are not limited to lung cancer cells, glioblastoma cells, and esophageal carcinoma cells.

A “control” is an alternative subject or sample used in an experiment for comparison purpose. A control can be “positive” or “negative”. For example, where the purpose of the experiment is to determine a correlation of the efficacy of a composition of the invention for the treatment for a particular type of disease or cancer, it is generally preferable to use a positive control (a compound or composition known to exhibit the desired therapeutic effect) and a negative control (a subject or a sample that does not receive the therapy or receives a placebo).

The terms “cancer,” “neoplasm,” and “tumor,” used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition.

A neoplasm is an abnormal mass or colony of cells produced by a relatively autonomous new growth of tissue. Most neoplasms arise from the clonal expansion of a single cell that has undergone neoplastic transformation. The transformation of a normal to a neoplastic cell can be caused by a chemical, physical, or biological agent (or event) that directly and irreversibly alters the cell genome. Neoplastic cells are characterized by the loss of some specialized functions and the acquisition of new biological properties, foremost, the property of relatively autonomous (uncontrolled) growth. Neoplastic cells pass on their heritable biological characteristics to progeny cells.

The past, present, and future predicted biological behavior, or clinical course, of a neoplasm is further classified as benign or malignant, a distinction of great importance in diagnosis, treatment, and prognosis. A malignant neoplasm manifests a greater degree of autonomy, is capable of invasion and metastatic spread, may be resistant to treatment, and may cause death. A benign neoplasm has a lesser degree of autonomy, is usually not invasive, does not metastasize, and generally produces no great harm if treated adequately.

Cancer is a generic term for malignant neoplasms. Anaplasia is a characteristic property of cancer cells and denotes a lack of normal structural and functional characteristics (undifferentiation).

A tumor is literally a swelling of any type, such as an inflammatory or other swelling, but modem usage generally denotes a neoplasm. The suffix “-oma” means tumor and usually denotes a benign neoplasm, as in fibroma, lipoma, and so forth, but sometimes implies a malignant neoplasm, as with so-called melanoma, hepatoma, and seminoma, or even a non-neoplastic lesion, such as a hematoma, granuloma, or hamartoma. The suffix “-blastoma” denotes a neoplasm of embryonic cells, such as neuroblastoma of the adrenal or retinoblastoma of the eye.

Histogenesis is the origin of a tissue and is a method of classifying neoplasms on the basis of the tissue cell of origin. Adenomas are benign neoplasms of glandular epithelium. Carcinomas are malignant tumors of epithelium. Sarcomas are malignant tumors of mesenchymal tissues. One system to classify neoplasia utilizes biological (clinical) behavior, whether benign or malignant, and the histogenesis, the tissue or cell of origin of the neoplasm as determined by histologic and cytologic examination. Neoplasms may originate in almost any tissue containing cells capable of mitotic division. The histogenetic classification of neoplasms is based upon the tissue (or cell) of origin as determined by histologic and cytologic examination.

“Inhibiting” tumor growth indicates a growth state that is curtailed compared to growth without any therapy. Tumor cell growth can be assessed by any means known in the art, including, but not limited to, measuring tumor size, determining whether tumor cells are proliferating using a ³H-thymidine incorporation assay, or counting tumor cells.

“Suppressing” tumor cell growth means any or all of the following states: slowing, delaying, and “suppressing” tumor growth indicates a growth state that is curtailed when stopping tumor growth, as well as tumor shrinkage.

The term “culturing” refers to the in vitro propagation of cells or organisms on or in media of various kinds. It is understood that the descendants of a cell grown in culture may not be completely identical (morphologically, genetically, or phenotypically) to the parent cell. By “expanded” is meant any proliferation or division of cells.

As used herein, the term “oncothermia” intends a modulated, deep electro-hyperthermia system, that is supportive, complementary therapy for tumor treatment. The method transfers energy using the principle of capactivie coupling (like a condenser) of radio waves of 13,56-MHz. Without being bound by theory, oncothermia is believed to work by utilizing the special absorption rate of the near-membrane extracellular liquid of the tumor. The tumor tissue has lower impedance than the surrounding tissues, so most of the energy is transmitted and absorbed by the cancerous lesion.

### Descriptive Embodiments

Applicant provides herein a method for inhibiting the growth of a solid tumor or treating cancer in a patient comprising, or alternatively consisting essentially of, or yet further consisting of administering to the patient an effective amount of Lipoplatin monotherapy in a first dose and a second dose, thereby inhibiting the growth of the solid tumor or treating cancer in the patient.

In one aspect, the first dose is administered on day 1 and the second dose is administered between 12 to 36 hours after completion of the first dose, or alternatively between 20 to 28 hours, or yet further between 23 and 25 hours after completion of the first dose.

The first dose/second dose therapy cycle can be repeated two or more times, at intervals comprising 4 to 40 days there between and any interval in between. Non-limiting examples of intervals include, without limitation, between 4 and 35 days there between, or alternatively between 6 and 10 days there between, or alternatively, between 8 and 16, or alternatively about every two weeks.

The effective amount is administered in a dose determined by the treating physician to provide the most therapeutic benefit to the patient and will vary with the patient, the cancer and the prior treatments and duration of the therapy. Non-limiting examples of first and second doses include a first dose comprising, or alternatively consisting essentially of, or yet further consisting of, from about 100 mg/m² to 300 mg/m² Lipoplatin and the second dose comprises from about 100 mg/m² to 300 mg/m² Lipoplatin, and any amount in between, e.g., from about 150 mg/m² to 250 mg/m² Lipoplatin and the second dose are comprise from about 150 mg/m² to 250 mg/m² Lipoplatin. In one aspect, the first and second dose comprise, or alternatively consist essentially of, or yet further consist of, about 200 mg/m² Lipoplatin.

The methods are useful to inhibit the growth of a solid tumor or treat a cancer from the group of metastatic or non-metastatic lung cancer, non-small cell lung cancer (NSCLC), breast cancer, Triple-negative breast cancer, gastric cancer, head and neck cancer, colon cancer, colorectal cancer, rectal cancer, mesothelioma, pancreatic cancer, brain cancer, (glioblastoma multiform or metastases) or ovarian cancer.

The method can be used as a first line, a second line or a third line therapy for the patient. In one aspect, the patient previously underwent surgical resection and/or radiotherapy. In a further aspect, the patient was previously treated with first line oxaliplatin therapy.

Any suitable route of administration is acceptable, and can be determined by the treating physician. Non-limiting examples include intravenously or by inhalation therapy.

The method can be repeated with varying cycles, e.g., two, three, four, five, six, seven, eight or more, and can be used as a maintenance therapy for a patient. For maintenance therapy, the time between the first and second therapy is about 21 days to 35 days there between, or alternatively every 26 days to 30 days there between or alternatively about every 5 to 6 weeks.

In a further aspect, the method further comprises, or alternatively consists essentially of, or yet further consists of, administering an effective amount of a second chemotherapeutic agent. Non-limiting examples of are described herein, e.g., one or more of oxaliplatin, paclitaxel, taxol, taxane, 5-Fluoropyrimidine (5-FU), vinorelbine or gemcitabine.

The methods are based on the following information. Lipoplatin was administered with varying treatment regimens. In one aspect, Lipoplatin was adminstered once weekly and in combination with a second agent, once every two weeks. Lipoplatin showed no renal toxicity and was as equally effective as cisplatin.

In one aspect of the invention, the second anticancer drug is a DNA alkylating agent which attaches an alkyl group to DNA. Such agents are well known in the art and are used to treat a variety of tumors. Non-limiting examples of a DNA alkylating agents are Nitrogen mustards, such as Mechlorethamine, Cyclophosphamide (Ifosfamide, Trofosfamide), Chlorambucil (Melphalan, Prednimustine), Bendamustine, Uramustine and Estramustine; Nitrosoureas, such as Carmustine (BCNU), Lomustine (Semustine), Fotemustine, Nimustine, Ranimustine and Streptozocin; Alkyl sulfonates, such as Busulfan (Mannosulfan, Treosulfan); Aziridines, such as Carboquone, ThioTEPA, Triaziquone, Triethylenemelamine; Hydrazines (Procarbazine); Triazenes such as Dacarbazine and Temozolomide; Altretamine and Mitobronitol.

In another aspect of the invention, the second anticancer drug is a platinum based compound which is a subclass of DNA alkylating agents. Such agents are well known in the art and are used to treat a variety of cancers, such as, lung cancers, head and neck cancers, ovarian cancers, colorectal cancer and prostate cancer. Non-limiting examples of such agents include Carboplatin, Cisplatin, Nedaplatin, Oxaliplatin, Triplatin tetranitrate, Satraplatin, Aroplatin, Lobaplatin, and JM-216. (see McKeage et al. (1997) J. Clin. Oncol. 201:1232-1237 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004).

“Oxaliplatin” (Eloxatin®) is a platinum-based chemotherapy drug in the same family as cisplatin and carboplatin. It is typically administered in combination with fluorouracil and leucovorin in a combination known as FOLFOX for the treatment of colorectal cancer. Compared to cisplatin the two amine groups are replaced by cyclohexyldiamine for improved antitumour activity. The chlorine ligands are replaced by the oxalato bidentate derived from oxalic acid in order to improve water solubility. Equivalents to Oxaliplatin are known in the art and include without limitation cisplatin, carboplatin, aroplatin, lobaplatin, nedaplatin, and JM-216 (see McKeage et al. (1997) J. Clin. Oncol. 201:1232-1237 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004).

In one aspect of the invention, the second anticancer drug is a topoisomerase inhibitor which is an agent that interferes with the action of topoisomerase enzymes (topoisomerase I and II). Topoisomerases are enzymes that control the changes in DNA structure by catalyzing the breaking and rejoining of the phosphodiester backbone of DNA. Such agents are well known in the art. Non-limiting examples of Topoisomerase I inhibitors include Campothecine derivatives including CPT-11/Irinotecan, SN-38, APC, NPC, camptothecin, topotecan, exatecan mesylate, 9-nitrocamptothecin, 9-aminocamptothecin, lurtotecan, rubitecan, silatecan, gimatecan, diflomotecan, extatecan, BN-80927, DX-8951f, and MAG-CPT as described in Pommier (2006) Nat. Rev. Cancer 6(10):789-802 and U.S. Patent Appl. No. 2005/0250854; Protoberberine alkaloids and derivatives thereof including berberrubine and coralyne as described in Li et al. (2000) Biochemistry 39(24):7107-7116 and Gatto et al. (1996) Cancer Res. 15(12):2795-2800; Phenanthroline derivatives including Benzo[i]phenanthridine, Nitidine, and fagaronine as described in Makhey et al. (2003) Bioorg. Med. Chem. 11(8):1809-1820; Terbenzimidazole and derivatives thereof as described in Xu (1998) Biochemistry 37(10):3558-3566; and Anthracycline derivatives including Doxorubicin, Daunorubicin, and Mitoxantrone as described in Foglesong et al. (1992) Cancer Chemother. Pharmacol. 30(2):123-125, Crow et al. (1994) J. Med. Chem. 37(19):3191-3194, and (Crespi et al. (1986) Biochem. Biophys. Res. Commun. 136(2):521-8.

In one aspect of the invention, the topoisomerase I inhibitors can be selected from the group of, but not limited to, Campothecine derivatives including CPT-11/Irinotecan, SN-38, APC, NPC, camptothecin, topotecan, exatecan mesylate, 9-nitrocamptothecin, 9-aminocamptothecin, lurtotecan, rubitecan, silatecan, gimatecan, diflomotecan, extatecan, BN-80927, DX-8951f, and MAG-CPT as described in Pommier (2006) Nat. Rev. Cancer 6(10):789-802 and US Patent Appl. No. 2005/0250854; Protoberberine alkaloids and derivatives thereof including berberrubine and coralyne as described in Li et al. (2000) Biochemistry 39(24):7107-7116 and Gatto et al. (1996) Cancer Res. 15(12):2795-2800; Phenanthroline derivatives including Benzo[i]phenanthridine, Nitidine, and fagaronine as described in Makhey et al. (2003) Bioorg. Med. Chem. 11(8):1809-1820; Terbenzimidazole and derivatives thereof as described in Xu (1998) Biochemistry 37(10):3558-3566; and Anthracycline derivatives including Doxorubicin, Daunorubicin, and Mitoxantrone as described in Foglesong et al. (1992) Cancer Chemother. Pharmacol. 30(2):123-125, Crow et al. (1994) J. Med. Chem. 37(19):3191-3194, and (Crespi et al. (1986) Biochem. Biophys. Res. Commun. 136(2):521-8, will be used in combination therapy with antibody based chemotherapy described above to treat patients identified with the appropriate genetic markers.

Irinotecan (CPT-11) is sold under the tradename of Camptosar®. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation.

In another aspect, some second anticancer agents inhibit Topoisomerase II and have DNA intercalation activity such as, but not limited to, Anthracyclines (Aclarubicin, Daunorubicin, Doxorubicin, Epirubicin, Idarubicin, Amrubicin, Pirarubicin, Valrubicin, Zorubicin) and Antracenediones (Mitoxantrone and Pixantrone).

In one aspect of the invention, Topoisomerase II inhibitors include, but are not limited to Etoposide and Teniposide.

In another aspect of the invention, the second anticancer drug is a dual topoisomerase I and II inhibitors selected from the group of, but not limited to, Saintopin and other Naphthecenediones, DACA and other Acridine-4-Carboxamindes, Intoplicine and other Benzopyridoindoles, TAS-103 and other 7H-indeno[2,1-c]Quinoline-7-ones, Pyrazoloacridine, XR 11576 and other Benzophenazines, XR 5944 and other Dimeric compounds, and Anthracenyl-amino Acid Conjugates as described in Denny and Baguley (2003) Curr. Top. Med. Chem. 3(3):339-353. In one aspect, they can be used in combination therapy with antibody based chemotherapy described above to treat patients identified with the appropriate genetic markers.

“Lapatinib” (Tykerb®) is an oncolytic dual EGFR and erbB-2 inhibitor. Lapatinib has been investigated as an anticancer monotherapy, as well as in combination with trastuzumab, capecitabine, letrozole, paclitaxel and FOLFIRI (irinotecan, 5-fluorouracil and leucovorin), in a number of clinical trials. It is currently in phase III testing for the oral treatment of metastatic breast, head and neck, lung, gastric, renal and bladder cancer. A chemical equivalent of lapatinib is a small molecule or compound that is a tyrosine kinase inhibitor or alternatively a HER-1 inhibitor or a HER-2 inhibitor. Several TKIs have been found to have effective antitumor activity and have been approved or are in clinical trials. Examples of such include, but are not limited to Zactima (ZD6474), Iressa (gefitinib) and Tarceva (erlotinib), imatinib mesylate (STI571; Gleevec), erlotinib (OSI-1774; Tarceva), canertinib (CI 1033), semaxinib (SU5416), vatalanib (PTK787/ZK222584), sorafenib (BAY 43-9006), sutent (SU11248) and leflunomide (SU101).

A biological equivalent of lapatinib is a peptide, antibody or antibody derivative thereof that is a HER-1 inhibitor and/or a HER-2 inhibitor. Examples of such include but are not limited to the humanized antibody trastuzumab and Herceptin.

In another aspect of the invention, the second anticancer drug is an antimetabolite agent which inhibits the use of a metabolite, i.e. another chemical that is part of normal metabolism. In cancer treatment, antimetabolites interfere with DNA production, thus cell division and growth of the tumor. Non-limiting examples of these agents are Folic acid based, i.e. dihydrofolate reductase inhibitors, such as Aminopterin, Methotrexate and Pemetrexed; thymidylate synthase inhibitors, such as Raltitrexed, Pemetrexed; Purine based, i.e. an adenosine deaminase inhibitor, such as Pentostatin, a thiopurine, such as Thioguanine and Mercaptopurine, a halogenated/ribonucleotide reductase inhibitor, such as Cladribine, Clofarabine, Fludarabine, or a guanine/guanosine: thiopurine, such as Thioguanine; or Pyrimidine based, i.e. cytosine/cytidine: hypomethylating agent, such as Azacitidine and Decitabine, a DNA polymerase inhibitor, such as Cytarabine, a ribonucleotide reductase inhibitor, such as Gemcitabine, or a thymine/thymidine: thymidylate synthase inhibitor, such as a Fluorouracil (5-FU).

Fluorouracil (5-FU) belongs to the family of therapy drugs call pyrimidine based antimetabolites. 5-FU is transformed into different cytotoxic metabolites that are then incorporated into DNA and RNA thereby inducing cell cycle arrest and apoptosis. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluroidine), 1-tetrahydrofuranyl-5-fluorouracil (ftorafur), Capecitabine (Xeloda), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamicheal (1999) The Oncologist 4:478-487.

Capecitabine and Tegafur are examples of chemical equivalents of 5-FU. It is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

Leucovorin (Folinic acid) is an adjuvant used in cancer therapy. It is used in synergistic combination with 5-FU to improve efficacy of the chemotherapeutic agent. Without being bound by theory, addition of Leucovorin is believed to enhance efficacy of 5-FU by inhibiting thymidylate synthase. It has been used as an antidote to protect normal cells from high doses of the anticancer drug methotrexate and to increase the antitumor effects of fluorouracil (5-FU) and tegafur-uracil. It is also known as citrovorum factor and Wellcovorin. This compound has the chemical designation of L-Glutamic acid N[4[[(2-amino-5-formyl1,4,5,6,7,8hexahydro4oxo6-pteridinyl)methyl]amino]benzoyl], calcium salt (1:1).

Examples of vincalkaloids, include, but are not limited to vinblastine, Vincristine, Vinflunine, Vindesine and Vinorelbine.

Examples of taxanes include, but are not limited to docetaxel, Larotaxel, Ortataxel, Paclitaxel and Tesetaxel. An example of an epothilone is iabepilone.

Examples of enzyme inhibitors include, but are not limited to farnesyltransferase inhibitors (Tipifarnib); CDK inhibitor (Alvocidib, Seliciclib); Proteasome inhibitor (Bortezomib); Phosphodiesterase inhibitor (Anagrelide); IMP dehydrogenase inhibitor (Tiazofurine); and Lipoxygenase inhibitor (Masoprocol).

Examples of tyrosine kinase inhibitors include, but are not limited to ErbB: HER1/EGFR (Erlotinib, Gefitinib, Lapatinib, Vandetanib, Sunitinib, Neratinib); HER2/neu (Lapatinib, Neratinib); RTK class III: C-kit (Axitinib, Sunitinib, Sorafenib); FLT3 (Lestaurtinib); PDGFR (Axitinib, Sunitinib, Sorafenib); and VEGFR (Vandetanib, Semaxanib, Cediranib, Axitinib, Sorafenib); bcr-abl (Imatinib, Nilotinib, Dasatinib); Src (Bosutinib) and Janus kinase 2 (Lestaurtinib).

PTK/ZK is a “small” molecule tyrosine kinase inhibitor with broad specificity that targets all VEGF receptors (VEGFR), the platelet-derived growth factor (PDGF) receptor, c-KIT and c-Fms. Drevs (2003) Idrugs 6(8):787-794. PTK/ZK is a targeted drug that blocks angiogenesis and lymphangiogenesis by inhibiting the activity of all known receptors that bind VEGF including VEGFR-1 (Flt-1), VEGFR-2 (KDR/Flk-1) and VEGFR-3 (Flt-4). The chemical names of PTK/ZK are 1-[4-Chloroanilino]-4-[4-pyridylmethyl]phthalazine Succinate or 1-Phthalazinamine, N-(4-chlorophenyl)-4-(4-pyridinylmethyl)-, butanedioate (1:1). Synonyms and analogs of PTK/ZK are known as Vatalanib, CGP79787D, PTK787/ZK 222584, CGP-79787, DE-00268, PTK-787, PTK-787A, VEGFR-TK inhibitor, ZK 222584 and ZK.

Additional examples of second chemotherapeutic agents and combination therapies include, but are not limited to amsacrine, Trabectedin, retinoids (Alitretinoin, Tretinoin), Arsenic trioxide, asparagine depleter (Asparaginase/Pegaspargase), Celecoxib, Demecolcine, Elesclomol, Elsamitrucin, Etoglucid, Lonidamine, Lucanthone, Mitoguazone, Mitotane, Oblimersen, Temsirolimus, and Vorinostat.

“FOLFOX” is an abbreviation for a type of combination therapy that is used to treat colorectal cancer. It includes 5-FU, oxaliplatin and leucovorin. Information regarding this treatment is available on the National Cancer Institute's web site, cancer.gov, last accessed on Jan. 16, 2008.

“FOLFOX/BV” is an abbreviation for a type of combination therapy that is used to treat colorectal cancer. This therapy includes 5-FU, oxaliplatin, leucovorin and Bevacizumab. Furthermore, “XELOX/BV” is another combination therapy used to treat colorectal cancer, which includes the prodrug to 5-FU, known as Capecitabine (Xeloda) in combination with oxaliplatin and bevacizumab. Information regarding these treatments are available on the National Cancer Institute's web site, cancer.gov or from the National Comprehensive Cancer Network's web site, nccn.org, last accessed on May 27, 2008. Examples of second chemotherapeutics or anticancer drugs include therapeutic antibodies include, but are not limited to anti-HER1/EGFR (Cetuximab, Panitumumab); Anti-HER2/neu (erbB2) receptor (Trastuzumab); Anti-EpCAM (Catumaxomab, Edrecolomab) Anti-VEGF-A (Bevacizumab); Anti-CD20 (Rituximab, Tositumomab, Ibritumomabi); Anti-CD52 (Alemtuzumab); and Anti-CD33 (Gemtuzumab), as well as biological equivalents thereof.

Bevacizumab is sold under the trade name Avastin by Genentech. It is a humanized monoclonal antibody that binds to and inhibits the biologic activity of human vascular endothelial growth factor (VEGF). Biological equivalent antibodies are identified herein as modified antibodies and those which bind to the same epitope of the antigen, prevent the interaction of VEGF to its receptors (FltOl, KDR a.k.a. VEGFR2) and produce a substantially equivalent response, e.g., the blocking of endothelial cell proliferation and angiogenesis.

In one aspect, the “chemical equivalent” means the ability of the chemical to selectively interact with its target protein, DNA, RNA or fragment thereof as measured by the inactivation of the target protein, incorporation of the chemical into the DNA or RNA or other suitable methods. Chemical equivalents include, but are not limited to, those agents with the same or similar biological activity and include, without limitation a pharmaceutically acceptable salt or mixtures thereof that interact with and/or inactivate the same target protein, DNA, or RNA as the reference chemical.

In one aspect, the “biological equivalent” means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody. An example of an equivalent Bevacizumab antibody is one which binds to and inhibits the biologic activity of human vascular endothelial growth factor (VEGF).

The methods disclosed herein are based, in part on a study that investigated toxicity and effectiveness when Lipoplatin is administered on two consecutive days, repeated every two weeks. A total of 21 patients with histologically- or cytologically-confirmed non-small cell lung cancer (NSCLC) were enrolled in the study. All but two patients, who had not been pretreated, had received one or two series of chemotherapy and some had undergone radiotherapy. Lipoplatin monotherapy was infused for 8 h the first and second days and repeated every 2 weeks with the aim of administering 6 cycles. The dose per day was 200 mg/m². Eight out of 21 (38.10%) patients had a partial response, 9 (42.86%) had stable disease and 4 (19.05%) had progressive disease. Results showed that there was no renal failure toxicity and no other adverse reactions apart from grade 1 myelotoxicity in only 2 patients who had been heavily pretreated, and grade 1 nausea/vomiting in 4 patients. Lipoplatin liposomal cisplatin is an agent with negligible toxicity and reasonably high effectiveness even when administered to pretreated patients with NSCLC.

The new agent, liposomal cisplatin (Lipoplatin), has been investigated in pre-clinical and clinical studies in recent years, and as yet there are more than 16 reports published in peer-reviewed journals (1). This agent was produced as a substitute for cisplatin and it has resulted in a reduction in toxicity compared to cisplatin, but with equal effectiveness. Liposomal cisplatin has been tested in patients with pancreatic, breast and mainly non-small cell lung cancer (NSCLC). The lipids of lipoplatin are composed of soy phosphatidyl choline (SPC-3), cholesterol, dipalmitoyl phosphatidyl glycerol (DPPG) and methoxy-polyethylene glycol-disteroyl phosphatidyl ethanolamine. The formulation was achieved by the formation of reverse micelles between cisplatin and DPPG under special conditions of pH, ethanol, ionic strength and other parameters. Lipoplatin has demonstrated a high increase of concentration in primary or metastatic tumors, with levels up to 10 to 50-fold higher than the uptake of the normal tissue adjacent to the tumor (22). Despite the number of publications related to lipoplatin, an analytical study evaluating the value of this agent with respect to toxicity and the modified two days of treatment is required.

Thus, one aim of the reported study of Lipoplatin, knowing its negligible toxicity, was to infuse this agent as monotherapy on days 1 and 2 every 2 weeks in pretreated and non-pretreated patients with NSCLC, and to determine the effectiveness of this treatment modification and whether toxicity is increased.

Based on the results, the method comprises administration of Lipoplatin to the patient at a dose of from about 100 mg/m2 to about 300 mg/m2 in a pharmaceutically acceptable carrier, such as 5% Dextrose or saline. Thus, this disclosure provides this composition as well.

The method also encompasses administration of the Lipoplatin composition at a dose from about 120 mg/m2 to about 250 mg/m2 every 7 days combined with low dose radiation therapy to the lesions in fractions on Days 2 and 3, or on Days 2, 3, 4, and 5. The method also encompasses administration of the Lipoplatin composition to treat locally advanced Triple-negative Breast Cancer using 200 mg/m2 intravenous (IV) on Days 1, 8, and 15 of each 28-day cycle. Patients can be restaged after 8 weeks and in case of partial response (PR) or complete response (CR) 8 more weeks of Lipoplatin is being delivered followed by maintenance therapy using 200 mg/m2 Lipoplatin every 4 weeks for life or until commencement of progressive disease (PD).

This disclosure also provides a method for inhibiting the growth of a solid tumor or treating cancer in a patient, comprising, or alternatively consisting essentially of, or yet further consisting of administering to the patient a first dose of Lipoplatin monotherapy, wherein the first dose comprises about 200 mg/m2 and a second dose to the patient of about 200 mg/m2 of Lipoplatin monotherapy about 24 hours after administration of the first dose. In one embodiment, the first dose and/or second dose is administered intravenously to the patient in a formulation comprising about 2 liters of a 5% Dextrose solution or saline. In a further aspect, the method further comprises, or alternatively consists essentially of, or yet further consists of, one or more treatment cycles comprising repeating the first dose and the second dose about every 14 days, after administration of the first dose. In one embodiment, the one or more treatment cycles comprises, or alternatively consists essentially of, or yet further consists of, at least 6 cycles of administration of the first dose and the second dose.

The methods are useful to inhibit the growth of a solid tumor or treat a cancer from the group of metastatic or non-metastatic lung cancer, non-small cell lung cancer (NSCLC), breast cancer, Triple-negative breast cancer, gastric cancer, head and neck cancer, colon cancer, colorectal cancer, rectal cancer, pancreatic cancer, mesothelioma, brain cancer, (glioblastoma multiform or metastases), brain cancers metastasized from a primary tumor outside the brain, or ovarian cancer. The method can be used as a first line, a second line or a third line therapy for the patient. In one aspect, the patient previously underwent surgical resection and/or radiotherapy. In a further aspect, the patient was previously treated with first line oxaliplatin therapy.

Any suitable route of administration is acceptable, and can be determined by the treating physician. Non-limiting examples include intravenously or by inhalation therapy.

For a comparison of the claimed therapies (see Table 2, below), Applicant notes Avastin® monotherapy, which showed marginal efficacy (Ogita et al, (2012) Pilot Phase II Trial of Bevacizumab Monotherapy in Nonmetastatic Castrate-Resistant Prostate Cancer. ISRN Oncol. 2012; 2012:242850. Epub 2012 Jun. 13).

In addition, a Japanese study showed an overall response rate of 9% using oxaliplatin monotherapy as second line in colorectal cancer and there were Grade 3 toxicities linked to it, notably neurophathy (Boku et al, (2007) Phase II study of oxaliplatin in japanese patients with metastatic colorectal cancer refractory to fluoropyrimidines. Jpn J Clin Oncol. 2007 June; 37(6):440-445) Objective responses were achieved in 20 and 24% of patients in 2 small trials of first-line oxaliplatin monotherapy and in about 10% of patients given oxaliplatin monotherapy as a second-line option (Wiseman et al, (2007) Oxaliplatin: a review of its use in the management of metastatic colorectal cancer. Drugs Aging. 1999 Jun.; 14(6):459-75). A response rate of 24% was achieved as first line oxaliplatin monotherapy in metastatic colorectal cancer patients with severe neurotoxicity in 10% of patients after 6 treatment cycles and in 50% after 9 cycles of an oxaliplatin dosage of 130 mg/m2 once every 3 weeks. A response rate of 4.5% was achieved in NSCLC using monotherapy with SPI-077 (a liposomal cisplatin by Alza) (White et al, (2006) Phase II study of SPI-77 (sterically stabilised liposomal cisplatin) in advanced non-small-cell lung cancer. Br J Cancer. 2006 Oct. 9; 95(7):822-8. Epub 2006 Sep. 12). Applicant's data shows the outstanding therapeutic use of Lipoplatin monotherapy. Since there is no cumulative toxicity, the Lipoplatin is suitable for maintenance therapy in cases where responders can benefit for life or till disease progression. Therefore, one could achieve conversion of some deadly forms of cancer into a chronic disease, at least for a group of responders.

Thus, in another aspect, this disclosure provides a method for inhibiting the growth of a solid tumor or treating cancer in a patient comprising, or alternatively consisting essentially of, or yet further consisting of, administering a first dose of Lipoplatin monotherapy, wherein the first dose comprises about 200 mg/m2 and a second dose of about 200 mg/m2 of Lipoplatin monotherapy about 4 weeks after administration of the first dose, thereby inhibiting the growth of the tumor or treating the patient. In one aspect, the first dose and/or second dose is administered intravenously to the patient in a formulation comprising about 2 liters of a 5% Dextrose solution or saline. In another aspect, the method further comprises, or alternatively consists essentially of, or yet further consists of one or more treatment cycles comprising, or alternatively consisting essentially of, or yet further consisting of, repeating the first dose and the second dose as a maintenance therapy for the patient for life or until disease progression of the cancer or solid tumor. This therapy is possible because Lipoplatin does not show cumulative toxicity thus allowing weekly doses for more than about 30 weeks. The same is not feasible with cisplatin given for up to 6 doses.

Further embodiments of the above noted methods are provided herein. In one aspect, the method comprises the administration to a patient of an effective amount of a combination of Lipoplatin monotherapy with an effective amount of low dose radiation therapy administered on the following about 2 of about 4 days for other cancers that can be treated with chemo radiation comprising but not limited to pancreatic cancer, brain tumors (glioblastoma multiform) or metastases from other primary tumors to the brain, ovarian cancer, breast cancer. In another aspect, the method comprises administration to a cancer patient of Lipoplatin monotherapy against locally advanced Triple-negative Breast Cancer at a dose of about 200 mg/m2 intravenous (IV) on days 1, 8, and 15 of each 28-day cycle. Patients are restaged after about 8 weeks and in case of partial response (PR) or complete response (CR) about 8 more weeks of lipoplatin is being delivered followed by maintenance therapy.

In another aspect, the method comprises administration of an effective amount of a combination Lipoplatin and paclitaxel for inhibiting or treating in nonsquamous-nonsmall cell lung cancer (ns-NSCLC) (50% of all lung cancers) in a patient. Applicant submits that this therapy achieves superiority over the gold standard cisplatin-paclitaxel and lowering all side effects of the acceptable combination for lung cancer.

In another aspect, the method comprises administration of an effective amount combination of Lipoplatin and paclitaxel to inhibit the growth of or treat in NSCLC in a patient. This method is superior over carboplatin and paclitaxel, considered the gold standard for NSCLC treatment in the United States and other countries of the world. It lowers all side effects, notably myelotoxicity of the carboplatin-paclitaxel regimen. In another aspect, the method is administration of an effective amount combination of Lipoplatin and gemcitabine in NSCLC which achieves superiority over cisplatin and gemcitabine, which is considered the gold standard for NSCLC treatment in Europe and other countries of the world and lowering all side effects, notably nephrotoxicity, neurotoxicity, gastrointestinal toxicity, ototoxicity and myelotoxicity of the cisplatin-gemcitabine regimen.

Also provided is a method for inhibiting the growth of a solid tumor or treating lung cancer, comprising, or alternatively consisting essentially of, or yet further consisting of administration of Lipoplatin monotherapy at a dose of about 120 mg/m2 to about 250 mg/m2, about every 7 days combined with an effective amount of low dose radiation therapy to the lesions in fractions on days 2, 3 or on Days 2, 3, 4, and 5.

Yet further provided is a method of inhibiting the growth of a solid tumor or treating cancer, comprising, or alternatively consisting essentially of, or yet further consisting of, administering an effective of amount of the combination of Lipoplatin and pemetrexed (Alimta, Eli Lily) to a nonsquamous-nonsmall cell lung cancer (ns-NSCLC) patient. This method can achieve superior results over the gold standard cisplatin-pemetrexed while lowering all side effects of the acceptable combination for ns-NSCLC in USA, Europe and most countries of the world.

Yet further provided is a method for inhibiting the growth of a solid tumor or treating cancer comprising, or alternatively consisting essentially of, or yet further consisting of, administration of an effective amount of Lipoplatin monotherapy or combinations of Lipoplatin with 5-fluorouracil and leucovorin or other chemotherapy drugs to patients with renal insufficiency. This group of patients is difficult to be treated as chemotherapy may result to life-threatening kidney damage. In a yet further aspect, a method is provided for inhibiting the growth of a solid tumor in a patient, comprising, or alternatively consisting essentially of, or yet further consisting of, administration of Lipoplatin monotherapy or combinations of Lipoplatin with other chemotherapy drugs known in the art to elderly patients (over 75 years of age). This group of patients is difficult to be treated with chemotherapy.

Yet further provided, Lipoplatin is an improved radiosensitizing agent as compared to cisplatin, carboplatin and oxaliplatin. Intravenous injection of Lipoplatin achieves targeting of human tumors, achieving a concentration up to 200-fold higher compared to platinum levels in the adjacent normal tissue.

In a further aspect, a method for inhibiting the growth of a solid tumor or for treating cancer, comprising or further consisting essentially of, or yet further consisting of, administration of an effective amount of Lipoplatin monotherapy or an effective amount of a combination of Lipoplatin with gemcitabine to treat pancreatic cancer achieving a significant survival advantage (over 30 one-year survival compared to 17% for gemcitabine alone).

Yet further provided, is a method for the treatment of mesothelioma, comprising, or alternatively consisting essentially of, or yet further consisting of, administration of an effective amount of Lipoplatin monotherapy or an effective amount of a combination of Lipoplatin with other cytotoxics.

The above methods are useful to inhibit the growth of a solid tumor or treat a cancer from the group of metastatic or non-metastatic lung cancer, non-small cell lung cancer (NSCLC), breast cancer, Triple-negative breast cancer, gastric cancer, head and neck cancer, colon cancer, colorectal cancer, rectal cancer, pancreatic cancer, mesothelioma, brain cancer, (glioblastoma multiform or metastases) or ovarian cancer. The method can be used as a first line, a second line or a third line therapy for the patient. In one aspect, the patient previously underwent surgical resection and/or radiotherapy. In a further aspect, the patient was previously treated with first line oxaliplatin therapy.

Any suitable route of administration is acceptable, and can be determined by the treating physician. Non-limiting examples include intravenously or by inhalation therapy.

While the patient is typically a human patient, the methods can also be practiced on suitable animal models (rats, mice and the like) and used to compare other therapeutic regimens with the disclosed methods and compositions.

Also provided is a method for inhibiting the growth of a brain tumor or treating a brain tumor in a subject, comprising, or alternatively consisting essentially of, or yet further consisting of, intra-arterial administration of an effective amount of Lipoplatin to the subject, thereby inhibiting the growth of the brain tumor or treating the brain tumor. In one aspect, the brain tumor is a glioblastoma multiform tumor or a tumor that has metastasized to the brain from a primary tumors outside the brain. In another aspect, the method further comprises administration of an effective amount of a one or more of a second therapeutic agent (as described above) or a drug that enhances penetration and transport of Lipoplatin across the blood-brain-barrier (BBB), low dose radiation or oncothermia. A non-limiting examples of drugs that enhances penetration and transport of Lipoplatin is temozolomid. In another aspect, the low dose radiation comprises one or more of an x-ray or a gamma knife. Intra-arterial administration of Lipoplatin can be combined with low dose radiation (x-rays, gamma knife, other sources) or oncothermia, with or without disruption of the blood-brain-barrier (BBB) with drugs such as temozolomide for the treatment of brain tumors (glioblastoma multiform and others) or metastases to the brain from other primary tumors.

This disclosure also provides a pharmaceutical Lipoplatin composition comprising, or alternatively consisting essentially of, or yet further consisting of, an effective amount of Lipoplatin to provide a dose of from about 100 mg/m2 to about 300 mg/m2 to a subject in a pharmaceutically acceptable carrier. In one aspect, the composition further comprises an effective amount of a drug that enhances transport of the Lipoplatin across the blood brain barrier, e.g., temozolomide Further provided is a kit comprising the Lipoplatin composition, alone or in combination with other therapeutic agents, and optionally instructions for performing the method as described herein.

### EXPERIMENTAL

### Experiment No. 1

### Materials and Methods

### Lipoplatin™

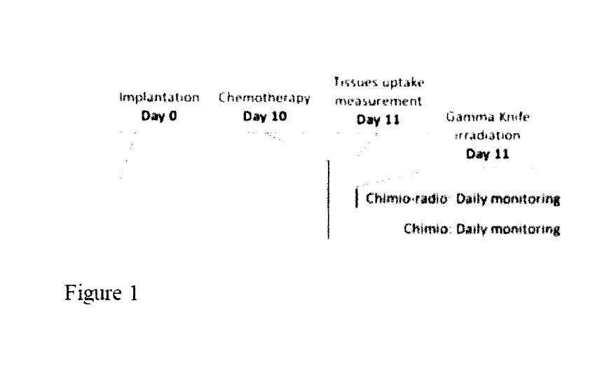

Lipoplatin™ is a therapeutic composition and its method of making are described in U.S. Pat. No. 7,393,478, incorporated by reference herein. Briefly, for the sake of completeness, Lipoplatin can be prepared by (step A) mixing cisplatin (in powder or other form) with DPPG (dipalmitoyl phosphatidyl glycerol) or other negatively-charged lipid molecules at a 1:1 to 1:2 molar ratio in at least a 30% ethanol, 0.1 M Tris HCl, pH 7.5 solution. Variations in the molar ratio between cisplatin and DPPG are also of therapeutic value targeting different tissues. In step (B), the composition is heated to 50° C. During steps A and B. the initial powder suspension, which tends to give a precipitate of the yellow cisplatin powder, is converted into a gel (colloidal) form; during steps A and B there is conversion of cisplatin to its aqua form (by hydrolysis of the chloride atoms and their replacement by water molecules bound to the platin) which is positively-charged and is the active form of cisplatin endowed with the antineoplastic activity; the aqua cisplatin is simultaneously complexed with the negatively-charged lipid into micelles in 30% ethanol. This cisplatin-DPPG electrostatic complex has already improved properties over free cisplatin in tumor eradication. (Step C) The properties of the complex (and of the final formulation after step D, see below) in passing through the tumor cell membrane after reaching its target are improved by addition of peptides and other molecules that give to the complex this property. (Step D) The cisplatin-DPPG micelle complex is converted into liposomes encapsulating the cisplatin-DPPG-monolayer (see FIG. 1 top of U.S. Pat. No. 7,393,478) or to other type of complexes by direct addition of premade liposomes followed by dialysis against saline and extrusion through membranes to downsize these to 100-160 nm in diameter (FIG. 1 bottom of U.S. Pat. No. 7,393,478). It is the lipid composition of added liposomes that determines the composition of the outer surface of our final cisplatin formulation. Variations in step (A) permit encapsulation of doxorubicin and other positively charged antineoplastic compounds. Addition of positively charged groups to neutral or negatively-charged compounds allows their encapsulation similarly into liposomes.

Lipoplatin Method of Administration.

The agent was infused for 8 h; the duration of time which has been established by other studies (1,22). As yet, no serious toxicity has been determined when lipoplatin is administered as monotherapy or in combination with another agent (23).

To date, lipoplatin has been administered once every week with no increase in side effects, while it is rarely administered once every 3 weeks. It has also been administered on days 1 and 8 and repeated on the 21st day (24-28). To determine the toxicity and effectiveness in the present study, the agent was administered for 2 consecutive days every 2 weeks.

### Eligibility Criteria

Patients.

Patients aged >18 years with a histologically- or cytologically-confirmed diagnosis of NSCLC stage IV with bidimensionally measurable disease were enrolled in the study. Two patients had not undergone prior chemotherapy or radiotherapy, while the remaining had pre-treatment of first- or second-line chemotherapy. Other eligibility criteria included a World Health Organisation (WHO) performance status (PS) of 0-2, a life expectancy of at least 3 months, adequate bone marrow reserve (granulocyte count, 1500 μl⁻¹; platelet count, 120000/μl⁻¹), normal renal function (serum creatinine concentration, <1.5 mg/dl) and liver function tests (total serum bilirubin, <3 mg/dl; provided that serum transaminases and serum proteins were normal), and normal cardiac function with no history of clinically unstable angina pectoris or myocardial infarction or congestive heart failure within the 6 months prior to the study. Patients with central nervous system involvement were eligible if they were asymptomatic. Patients with active infection, malnutrition or a second primary tumor (with the except of a non-melanoma skin epithelioma or in situ cervix carcinoma) were excluded from the study. The study was approved by our institutional review boards and all patients provided written informed consent to participate.

Treatment Plan.

Patients were treated on an outpatient basis. Lipoplatin was administered on days 1 and 2, and every 2 weeks again for two days. The treatment was designed to administer 6 courses at minimum (each course involved the two consecutive days of administration). The dose was 200 mg/m² per day based on the maximum tolerated dose defined by a previous phase I study (23). Lipoplatin was produced by Regulon Inc. (Mountain View, Calif., USA) and Regulon AE (Alimos, Athens, Greece). The Lipoplatin infusion time was 8 h. According to pharmacokinetics, there is slow renal excretion whereby 40% of the drug is excreted in 3 days (29). Premedication involved 8 mg of ondansetron and 8 mg of dexamethasone. In cases of severe myelotoxicity, the treatment would have been postponed for 3-7 days. Toxicities were graded according to the WHO guidelines (30).

Patient Evaluation.

Pretreatment evaluation included complete medical history and physical examination, full blood count, including differential leukocyte and platelet counts, a standard biochemical profile (and creatinine clearance when necessary), electrocardiogram, chest X-ray, ultrasound of the upper abdomen and computed tomography (CT) scans of the chest, upper and lower abdomen. Additional imaging studies were performed upon clinical indication. Full blood counts were performed weekly. In cases of grade 3 and 4 neutropenia or thrombocytopenia, full blood counts were evaluated daily.

A detailed medical and physical examination was completed prior to each course. Biochemical tests, ECG and chest X-rays were performed every 4 weeks and CT scans were performed at the end of the 3rd cycle.

Definition of Response.

For the assessment of response, imaging-based evaluation was used. A complete response (CR) was considered to be the disappearance of all measurable disease confirmed at 6 weeks at the earliest. Partial response (PR) was a 30% tumor decrease, while stable disease (SD) was determined if neither the PR nor the progressive disease (PD) criteria were met; indicating a 20% increase in tumor burden in PD, but not for CR, PR or SD documented before increased disease. Response data were based on the response evaluation criteria in solid tumors (RECIST) (31). A two-step deterioration in performance status (PS), a >10% loss in pretreatment weight or increasing symptoms, did not constitute progression of the disease. However, the progression of these complaints was followed by a new evaluation of the extent of the disease. All responses had to be maintained for at least 6 weeks and be confirmed by an independent panel of radiologists.

Statistical Analysis.

Simon's two-stage minimax design was used for the calculation of the sample size. The significance level was set at 5% and the power at 90%. The low response probability was set at 20% and the level of useful activity at 40%. In the first stage, 15 patients were enrolled in the study. If at least five responses were observed, more patients were recruited. For the main objective, which was to determine the toxicity, 20 patients were considered to be sufficient.

The primary endpoints of the study were to determine the toxicity (adverse reactions) and tumor responsiveness. The duration of the response was calculated from the day of the first demonstration of response until PD. Overall survival (OS) was calculated from the day of enrollment until the end of the study or death. Time to tumor progression was calculated from day of entry into the study until documented PD. The estimation of survival distribution was calculated by the Kaplan-Meier method.

### Results

Patient Characteristics.

A total of 21 patients were recruited into the study between January 2011 and November 2011. According to the statistical design, this number of patients was considered adequate with respect to the objective of the study.

The 21 patients comprised 20 males and 1 female (Table I). Of the 21 patients, 19 patients had adenocarcinoma and 2 had squamous cell carcinoma. The majority of patients had low differentiation disease. Metastasis was observed in the liver, bones, other lung, adrenal gland and brain in 3 patients (the latter had undergone radiation therapy).

Compliance with Treatment.

Seventy-five cycles were administered in total (150 infusion days). The median number of cycles was 4 and the range was 1-6. No patient had treatment delay due to myelotoxicity or other side effects; only 2 patients had a one-week delay due to a respiratory infection which was treated with antibiotics. Drug dose reduction was not required due to adverse reactions and no growth factor was administered. At the time of analysis, 2 patients had received the treatment as first-line, 10 as second-line and 9 as third-line. Nine patients remained alive and well at the end of the study, 9 patients succumbed to the disease, 2 patients succumbed to a heart attack 2 months after the end of the treatment and 1 patient was lost to follow-up.

Response Rate and Survival.

Survival was evaluated on an intention-to-treat basis. There was no CR in the 21 evaluable patients. Eight (38.10%) patients achieved a PR, 9 (42.86%) had SD and 4 (19.05%) had PD (Table II). Among the responders, 2 patients underwent first-line treatment, 5 second-line and 1 patient had third-line treatment. Four patients with SD had second-line treatment and 5 had third-line. Among the nonresponders (PD), 1 patient had second-line treatment and 3 patients had third-line. No PD was observed in any of the patients who achieved a PR for 4-6 months after treatments, and the median time of survival of the 8 patients with a PR was 7 months, range 3-10+ months. It is worth mentioning that in two patients with a minor response the tumor biopsy examination after treatment was full of necrotic cells.

Toxicity.

All 21 patients were evaluable for toxicity. There was no myelotoxicity (neutropenia, thrombocytopenia or anemia) in 19 of the 21 patients. Two (9.52%) patients experienced grade 1 myelotoxicity, but these patients had been heavily pretreated. Grade 1 nausea and vomiting on the first or second day after treatment was observed in 4 (19.05%) patients. Grade 1 fatigue and peripheral neuropathy were observed in 3 (14.29%) patients. No alopecia was observed. During the time of the drug infusion, temporary myalgia was observed in 5 patients, but it lasted for only 5-10 min. Notably, no renal toxicity (blood urea-serum creatinine were not increased) was detected, even after the 6th treatment course.

### Discussion

This study presents a new type of liposomal administration, with the infusion of the drug on days 1 and 2, with repetition every 2 weeks. It was determined that this agent can easily be administered for two consecutive days without causing serious adverse reactions, and particularly without causing renal toxicity. The results showed that patients were able to tolerate 4 lipoplatin infusions in 2 weeks. The determination in this study of the negligible toxicity of lipoplatin indicates that it may be administered even as first-line treatment to patients with NSCLC who would not be able to tolerate the serious adverse reactions caused by other agents. Patients with lung cancer who may have renal insufficiency, cardiac problems or other chronic disease could be selected for this modified two consecutive days of treatment every 2 weeks. The results of the present study and those of another study presented at the 2011 ASCO Congress may provide enough data concerning the choice of treatment for patients with NSCLC (32). If lipoplatin is combined with another agent, such as paclitaxel, vinorelbine or gemcitabine, there is no requirement for lipoplatin dose reduction.

The value of liposomal cisplatin in clinical practice, mainly in patients with NSCLC, may gradually establish it as a substitute for cisplatin. In this study, the effectiveness of lipoplatin was reasonably high, even in pretreated patients with NSCLC.

In the present study, a two-day treatment of liposomal cisplatin has been investigated and negligible toxicity determined. Renal, myelotoxicity (apart from grade 1) and other side effects were not observed, even with the administration of the drug at the maximum tolerated dose on the first and second days. Effectiveness remained high even in pretreated patients with NSCLC.

TABLE I
Characteristics of the 21 patients included in the study.

| | No. of patients | % |
|---|---|---|
| Patients enrolled | 21 | 100 |
| Patient evaluable | 21 | 100 |
| Gender | | |
| Male | 20 | 95.24 |
| Female | 1 | 4.76 |
| Age (years) | | |
| Median | 64 | |
| Range | 38-76 | |
| Disease stage | | |
| IIIA | 0 | 0 |
| IIIB | 0 | 0 |
| IV | 21 | 100 |
| Histology | | |
| Adenocarcinoma | 19 | 90.48 |
| Squamous cell carcinoma | 2 | 9.52 |
| Performance status | | |
| 0 | 6 | 28.57 |
| 1 | 7 | 33.33 |
| 2 | 8 | 38.10 |

TABLE II
Response rates.

| | 2-day treatment of lipoplatin | | |
|---|---|---|---|
| | every 2 weeks | No. of patients | % |
| CR | | 0 | 0 |
| PR | 1^{st} line | 2 | 38.10 |
| | 2^{nd} line | 5 | |
| | 3^{rd} line | 1 | |
| SD | 2^{nd} line | 4 | 42.86 |
| | 3^{rd} line | 5 | |
| PD | 2^{nd} line | 1 | 19.05 |
| | 3^{rd} line | 3 | |

CR, complete response;
PR, partial response;
SD, stable disease;
PD, progressed disease

### Experiment No. 2

The present study compares three different routes of administration (IV, IA accompanied or not with blood brain barrier disruption (BBBD) for five platinum drugs (cisplatin, oxaliplatin, carboplatin, Lipoplatin™, Lipoxal™) alone and in combination with focused radiation delivered by a Gamma Knife Tumor uptake, toxicity and improvement of the mean survival of Fischer rats implanted in their brain with the F98 glioma tumor were measured. Platinum compounds were chosen for their known radiosensitizing ability that is attributed to an enhancement of the production of DNA single and double-strand breaks. To better exploit their radiosensitizing effect while trying to prevent adverse effects, liposomal formulations of cisplatin and oxaliplatin, which are respectively Lipoplatin™ and Lipoxal™ were also tested.

### Chemicals

Carboplatin and oxaliplatin were obtained respectively from Novopharm (Anjou, Qc, Canada) and Sanofi-Avantis (Laval, Qc, Canada). Cisplatin was purchased from Sigma-Aldrich (Oakville, ON, Canada). Lipoplatin™ and Lipoxal™ were generously provided by Regulon Inc (Athens, Greece).

### Cell Line and Culture Conditions

The rat F98 Fischer glioma model was chosen since it was shown to adequately reproduce the behaviour of human glioblastoma. The F98 cell line was obtained from American Type Culture Collection (Manassas, Va., USA) and tested negative for the MAP assay by Charles River Laboratories (Wilmington, Mass., USA). Cells preparation and maintenance are described by Blanchard et al. (2006) Can J Neurol Sci, 33:86-91.

### Animal Experiments

For all procedures (implantation, chemotherapy, radiotherapy and euthanasia) male Fischer rats (Charles River Laboratories, Saint-Constant, Qc, Canada) were anesthetised with an intraperitoneal injection of ketamin/xylazine (87/13 mg/mL) at 1 mL/kg. The experimental protocol was approved by the institutional ethical committee and conformed to regulations of the Canadian Council on Animal Care. A diagram of the overview of the experimental strategies used is shown in FIG. 1.

### F98 Glial Cells Implantation in Fischer Rat Brain

For the implantation procedure, confluent F98 cells were suspended in non-supplemented warm MEM at a concentration of 2000 cells/pt. The implantation (10 000 cells in 54) was performed as described by Blanchard et al. (2006) Can J Neurol Sci, 33:86-91.

### Routes of Drug Administration

Ten days after implantation F98 glioma cells, platinum compounds were administrated. Equivalent doses of platinum compounds to those used in humans were established with respect to the body surface area (BSA), which is determined as 0.04 m2 for rats weighting 250 g. Platinum doses used in this study were: carboplatin 5 mg, oxaliplatin 3 mg, cisplatin 3 mg, Lipoplatin™ 3 mg (of cisplatin) and Lipoxal™ 3 mg (of oxaliplatin). Free platinum was diluted in 1 mL of 5% dextrose solution (Baxter, Toronto, ON, Canada). Lipoplatin™ and Lipoxal™ were used without dilution at a concentration of 3 mg platinum/mL.

The IV injections were performed via the tail vein over two minutes. Regarding the groups of animal injected IA, the drugs were infused in the right internal carotid artery in a retrograde manner via the external carotid as described by Fortin et al. (2004) Can J Neurol Sci., 31:248-253 and Charest G, et al. (2012) Treatment: Bypassing the Toxicity of Platinum Compounds by Using Liposomal Formulation and Increasing Treatment Efficiency with Concomitant Radiotherapy Int J Radiat Oncol Biol Phys. 2012; Epub ahead of print. A solution of 1 mL of platinum formulation was injected over 20 min. Temporary disruption of the blood brain barrier (BBBD) was obtained following the same surgical procedure as for the IA procedure. Previously, the opening of BBB by injecting IA in the carotid was quantified and optimized by a solution of mannitol. A MRI scanner for animals was used to follow after injection of mannitol the temporal opening of BBB.

The permeability of the BBB was increased early after injection of mannitol and remained open for at least the first 30 min. (Blanchette M. et al. (2009) Neurosurgery. 65:344-550. Drugs tested in the present study were injected during this time frame. Before platinum injection, a warm (37° C.) solution of mannitol 25% was infused in the right internal carotid artery in a retrograde manner via the external carotid at a rate of 7.20 mL/min for 30 s as described elsewhere. (Blanchette M, (2011) Methods Mol Biol. 686:447-463 and Blanchette M, et al. (2009) Neurosurgery. 65:344-550. Beginning three min after the BBBD, the drugs were infused over 20 min by the same catheter used for the mannitol injection. After IA infusion, the external carotid was sacrificed and the neck of the animal was closed by sutures.

### Platinum Uptake in Tumor and Brain Tissue

Animals (n=3 to 4 animals per group) were implanted with the F98 glioma cells at day zero, injected with platinum compounds at day 10 and euthanized 24 h later. Brains were removed by craniotomy and promptly cut in three sections with a brain matrix (WPI, RBMA-300C, Sarasota, Fla.) as describe elsewhere. Charest G. et al. (2012) Treatment: Bypassing the Toxicity of Platinum Compounds by Using Liposomal Formulation and Increasing Treatment Efficiency with Concomitant Radiotherapy Int J Radiat Oncol Biol Phys. 2012; Epub ahead of print. Tumor section of a thickness of 3.5 mm was standardized in the right hemisphere between slots 2 and 4 of the brain matrix (starting from frontal position), the tumor implantation point being located in the middle of slot 3. The left hemisphere (contralateral section) and healthy right hemisphere (adjacent tissue) were also isolated. Fresh tissue samples were rapidly weighed and solubilised in 10% nitric acid, 30% hydrogen peroxide and sonicated until homogenization. Samples were then analysed for platinum concentration by Inductively Coupled Plasma Mass Spectrometer (ICP-MS) (ELAN DRC-II, PerkinElmer, Woodbridge, ON, Canada).

### Gamma Knife Irradiation of Brain Tumor

Twenty four hours after chemotherapeutic treatments (platinum compounds and sham), rats (n=8-12 animals per group, except for cisplatin where n=4 animals) were anesthetised and positioned in our home made stereotactic frame²¹ designed for the Gamma Knife 4C and later adapted²² for the Gamma Knife PERFEXION (Elekta Instruments AB, Norcross, Ga., USA). The 8 mm collimators were used to deliver the radiation treatment (15 Gy with a dose rate of approximately 2.8 Gy/min) at predetermined coordinates targeting the tumor which had a diameter of about 4 mm. See Blanchard J. et al. (2006) Can J Neurol Sci., 33:86-91. Fractionation with a daily radiation dose of 2 Gy was deemed impractical for our experiments, since such a protocol requires repetitive animal anaesthesia, which leads cumulatively to important toxic effects. Therefore, the brain tumors were irradiated with a single dose of 15 Gy which is approximately equivalent to a typical protocol of 25 daily fractions of 2 Gy.

Control animals (sham) received the same surgical procedures as treated animals and 1 mL of 5% dextrose (vehicle for platinum drugs) was infused as performed for animals treated with platinum compounds.

### Evaluation of Mean Survival Time

Monitoring including weight measurement, mobility, coordination, loss of self-grooming (periocular secretion accumulation) and landing ability was performed on a daily basis. In agreement with the ethical committee regulations, the experimental endpoint for survival was established when the animals lost a maximum of 30% of their initial weight or when one of the monitored function reached a score of 1/10. Usually, a quasi-complete lethargy (and apathy) of the animals was observed at the endpoint. At this point, animals were anesthetised and 4% paraformaldehyde (PFA) was infused by intracardiac route to fix the brain tissue. The brain was removed by craniotomy to corroborate the presence of tumor and to be kept in PFA for future analysis.

### Statistics

Data of brain tissue accumulation were analysed by a Student's t-test to compare two treatments together and by ANOVA for more than two groups. For the survival study, data were analysed by the Quartile method before doing Kaplan-Meier survival curves which were analysed by Log-Rank test. P values under 0.05 were considered statistically significant.

### Results

### Drugs Accumulation in Nucleus and Cytoplasm of Tumor Cells

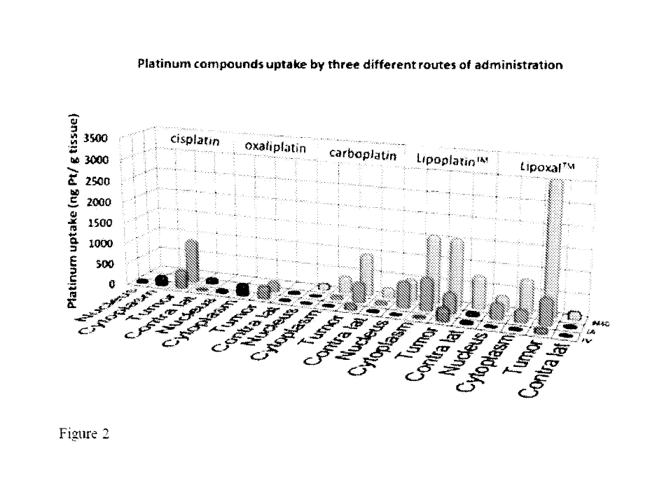

When the IV route of administration was used, the uptakes of carboplatin, Lipoplatin™ and Lipoxal™ in the nucleus of cancer cells were very low (˜4 ng platinum/g tissue), whereas the accumulations of cisplatin and oxaliplatin were significantly more substantial (P<0.03) with 67±14 and 78±8 ng platinum/g tissue respectively. All these drugs were also distributed preferentially in the cytoplasm (Experiment No. 2, Table 1 and FIG. 2).

Table 1, Experiment No. 2: Effect on uptake, mean surviving time and toxicity of glioblastoma bearing rats treated with five platinum compounds injected by three different routes of administration, with and without radiation.

TABLE 1
Accumulation of platinum drugs in nucleus
and cytoplasm of tumor cells.

| Drugs | Administration | Nucleus^{a} | Cytoplasm^{a} |
|---|---|---|---|
| Cisplatin | IV | 67 ± 14 | 251 ± 48 |
| | IA | ND | ND |
| | IA + BBBD^{b} | ND | ND |
| Oxaliplatin | IV | 78 ± 8 | 292 ± 128 |
| | IA | ND | ND |
| | IA + BBBD | ND | ND |
| Carboplatin | IV | 0.5 ± 0.8 | 13 ± 4 |
| | IA | 9 ± 7 | 84 ± 61 |
| | IA + BBBD | 160 ± 85 | 398 ± 191 |
| Lipoplatin ™ | IV | 4 ± 1 | 46 ± 8 |
| | IA | 613 ± 185 | 749 ± 99 |
| | IA + BBBD | 509 ± 332 | 1584 ± 684 |
| Lipoxal ™ | IV | 4 ± 1 | 32 ± 6 |
| | IA | 363 ± 78 | 285 ± 4 |
| | IA + BBBD | 365 ± 193 | 799 ± 394 |

^{a}Values in ng of platin/g tissue ± SD
^{b}BBBD = Blood Brain Barrier Disruption

Administration through the IA route largely increased the concentration of drugs in tumor cells. Accumulations of the liposomal formulations Lipoplatin™ and Lipoxal™ were increased by 118 to 152 times compared to the values obtained with the IV route. It is noteworthy that although carboplatin administrated by IA reached higher levels in nucleus and cytoplasm than measured after IV injection, this drug was still accumulated at lower levels than cisplatin and oxaliplatin injected IV.

IA administration was then combining to a temporary opening of the BBB to further expose tumor cells to the drugs. For Lipoplatin™ and Lipoxal™, opening of the BBB did not further increase their accumulation in nucleus of tumor cells (P=0.32 and 0.49 respectively). An increase of 2-fold was observed only in the cytoplasm for these drugs. Conversely, it was worth it to open the BBB for carboplatin whose concentration in the nucleus of tumor cells was promoted by 18-fold, while a 4.7-fold increase was measured in the cytoplasm (nucleus IA=9±7, nucleus BBBD=160±85, cytoplasm IA=84±61, cytoplasm BBBD=398±191).

### Drugs Accumulation in Tumor and Contra Lateral Brain

The impact of the routes of administration on the distribution of these platinum drugs between the tumor and the healthy contra lateral brain was measured (Table 1, FIG. 2). For all drugs, a preferential accumulation in the tumor area was measured, whatever the route of administration used. The IA route improved both the tumor uptake and specificity for carboplatin, Lipoplatin™ and Lipoxal™, but not for cisplatin. Surprisingly, the tumor uptake of oxaliplatin was not modified when this drug was injected by IA.

Temporal disruption of the BBB increased by 2 to 5-fold the drug accumulation in tumor, the highest improvement being observed with the liposomal formulations. Regarding the contra lateral brain, administration through IV or IA resulted in a similar and modest drug uptake for all the drugs, excepted for cisplatin. However, disruption of BBB has promoted by 3.4 to 10-fold the distribution of carboplatin, Lipoplatin™ and Lipoxal™ in the contra lateral brain.

Cisplatin and oxaliplatin were not evaluated for IA+BBBD since they were too toxic for the animals when administered.

### Anti-Cancer Effect and Toxicity of the Platinum Compounds

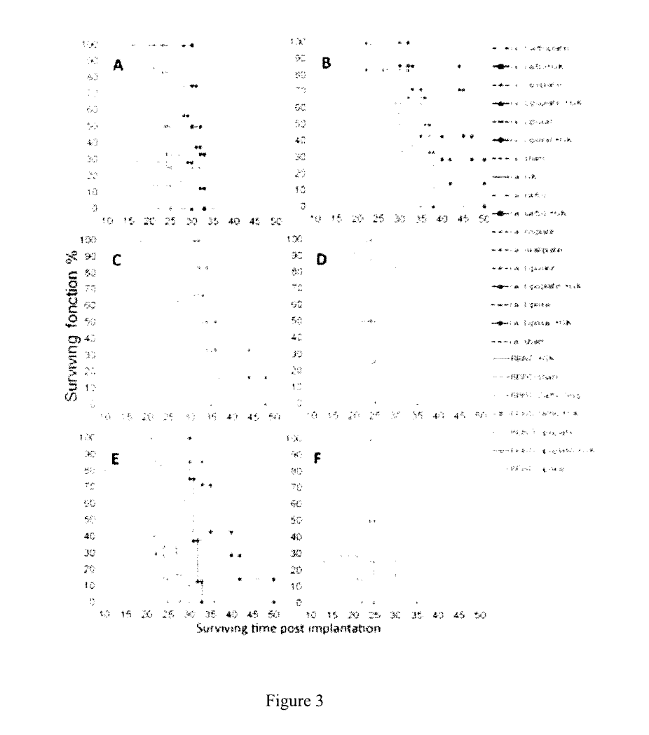

Administration by IV for all the platinum drugs tested did not lead to any therapeutic effect as measured by the mean survival time of Fischer rats implanted with the F98 brain tumor (Experiment No. 2, Table 1 and FIG. 3). Carboplatin, oxaliplatin, Lipoplatin™ and Lipoxal™ did not significantly increase the mean survival time of the animals compared to the sham group (22.6±1.2 days). Worse still, injection of cisplatin has shorten mean survival time to 18.1±0.9 days (P=0.012, compared to IV sham group), suggesting that this drug is too toxic for the animal even when injected IV.

The toxic effect of cisplatin was amplified when administered by the IA route. The drug was injected 10 days after the implantation of the F98 tumor, and a severe apathy was observed 3 days later resulting in a mean survival time of 13.3±0.1 days, which is much shorter than the sham group (22.5±0.6 days, P=0.001) (FIG. 3B). Regarding oxaliplatin, its administration through IA did not result in any improvement of the mean survival time (22.0±4.7 days vs 22.5±0.6 days for sham group, P=0.98).

Drug administration by IA was beneficial for the animals treated with carboplatin, Lipoplatin™ and Lipoxal™. The mean survival time using these drugs was improved from 6.7 to 8.5 days compared to the sham group (P<0.002).

Drug injection by IA was then combined to a temporary opening of the BBB to further increase exposure of brain tumor to these drugs. Assay with cisplatin and oxaliplatin was not conducted considering their toxicity or lack of anti-cancer effect with this animal model. Opening of the BBB was not beneficial for all the three other platinum drugs. For the liposomal formulation of cisplatin, Lipoplatin™, the mean survival time were similar with or without opening of the BBB (IA=29.2±1.8 days; IA+opening BBB=29.4±6.1 days, P=0.74). Opening of the BBB was detrimental for animals treated with Lipoxal™. The mean survival time of these animals was shorter but not significant than the group injected IA without opening of the BBB (IA=30.1±2.9 days; IA+opening BBB=21.1±12.9 days, P=0.99). It is noteworthy that for these animals injected IA with Lipoxal™ combined to opening of the BBB, an important apathy was observed in the first 24 h after treatment. When the animals were able to overcome this initial acute toxicity, their mean survival time was extend to 39 days compared to 33 days with IA. Only animals treated with carboplatin have taken advantage to the administration procedure combining IA injection and opening of the BBB, but the improvement was not significant (AI=31.0±3.6 days; IA+opening BBB=33.7±2.0, P=0.35).

Concomitant Treatment with Radiation

Irradiation (IR) of the F98 tumor without platinum compounds increased the mean survival time of the animals from 22.9±3.2 days (sham group) to 29.7±1.4 days (sham group+IR)/When platinum compounds were administrated IV and combined with radiation, only the group treated with Lipoxal™ showed a modest significant increase in the mean survival time from 29.7±1.4 days to 31.4±0.5 days (P=0.045).

IA injection combined to tumor irradiation was beneficial for animals treated with Lipoxal™ and carboplatin, but not with Lipoplatin™ (FIG. 3B). The mean survival time was increased by 3.9 days with Lipoxal™ (sham group+IR=34.0±6.1 days vs Lipoxal™+IR=37.9±6.7 days, P=0.40), but only the combination of carboplatin and IR showed a significant increase compared to the sham IR group with a 10.7 days increase of the mean survival time (44.7±6.1 days, P<0.004).

A temporary opening of the BBB has resulted in important toxicity for animals injected with Lipoxal™. Consequently, combination with tumor irradiation was not conducted. Regarding treatment with Lipoplatin™, no improvement of the mean survival time was measured in the irradiated animals (sham group+IR=34.5±2.2 days vs Lipoplatin+IR=33.2±1.8 days, P=0.14). A small but not significant benefit was measured only with carboplatin (sham group+IR=34.5±2.2 days vs carboplatin+IR=38.0±6.4 days, P=0.33).

When the irradiated groups of animals were analyzed according to the administration route, tumor irradiation combined to the IA route increased the mean survival time for each drug tested compared to the IV route (P<0.012). For the drugs tested (Lipoplatin™ and carboplatin), IA administration associated with the opening of the BBB has increased the mean survival time of the animals when compared to the groups injected IV (P<0.004). Finally, the combination of IA administration and opening of the BBB did not significantly improve the anti-cancer activity of Lipoplatin™ and carboplatin, compared to the IA groups (P>0.077).

TABLE 2
Accumulation of platinum drugs in brain
tumor and healthy contra lateral brain.

| Drugs | Administration | Tumor^{a} | Contra lateral brain^{a} |
|---|---|---|---|
| Cisplatin | IV | 453 ± 189 | 39 ± 14 |
| | IA | 1032 ± 173 | 148 ± 40 |
| | IA + BBBD^{b} | ND | ND |
| Oxaliplatin | IV | 310 ± 180 | 21 ± 5 |
| | IA | 249 ± 123 | 39 ± 11 |
| | IA + BBBD | ND | ND |
| Carboplatin | IV | 145 ± 27 | 27 ± 2 |
| | IA | 469 ± 241 | 70 ± 48 |
| | IA + BBBD | 987 ± 621 | 242 ± 59 |
| Lipoplatin ™ | IV | 321 ± 17 | 62 ± 8 |
| | IA | 473 ± 262 | 76 ± 9 |
| | IA + BBBD | 1547 ± 622 | 762 ± 219 |
| Lipoxal ™ | IV | 136 ± 29 | 20 ± 3 |
| | IA | 608 ± 337 | 41 ± 13 |
| | IA + BBBD | 3061 ± 642 | 198 ± 129 |

^{a}Values in ng of platin/g tissue ± SD
^{b}BBBD = Blood Brain Barrier Disruption

Thus, it should be understood that although the present disclosure has been specifically disclosed by preferred embodiments and optional features, modification, improvement and variation of the disclosure embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Stathopoulos G P and Boulikas T: Lipoplatin formulation review article. J Drug Deliv: Aug. 29, 2011 (Epub ahead of print).
2. Non-Small-Cell Lung Cancer Collaborative Group: Chemotherapy in non-small-cell lung cancer, a meta-analysis using updated data on individual patients from 52 randomised clinical trials. BMJ 311: 899-909, 1995.
3. Shepherd F A, Dancey J, Arnold A, et al: Phase II study of pemetrexed disodium, a multitargeted antifolate and cisplatin as first line therapy in patients with advanced non-small-cell lung cancer. Cancer 92: 595-600, 2001.
4. Ardizzoni A, Boni L, Tisseo M, et al: Cisplatin versus carboplatin-based chemotherapy in first line treatment of advanced non-small-cell lung cancer: an individual patient data metaanalysis. J Natl Cancer Inst 99: 847-857, 2007.
5. Aabo K, Adams M, Adnitt P, et al: Chemotherapy in advanced ovarian cancer: four systemic meta-analysis of individual patient data from 37 randomized trials. Br J Cancer 78: 1479-1487, 1998.
6. Taylor A E, Wiltsaw E, Gore M E, et al: Long-term follow-up of the first randomized study of cisplatin versus carboplatin for advanced epithelial ovarian cancer. J Clin Oncol 12: 2066-2070, 1994.
7. Pignon J P, Bourhis J, Domange C and Designé L: Chemotherapy added to locoregional treatment for head and neck squamous cell carcinoma: three meta-analyses of updated individual data. Lancet 355: 949-955, 2000.
8. Einform L H, Williams S D, Loehrer P T, et al: Evaluation of optimal duration of chemotherapy in favourable-prognosis disseminated germ cell tumors: a Southeastern Cancer Study Group Protocol. J Clin Oncol 7: 387-391, 1989.
9. Kaufman D, Raghavan D, Carducci M, et al: Phase II trial of gemcitabine plus cisplatin in patients with metastatic urothelial cancer. J Clin Oncol 18: 1921-1927, 2000.
10. Rosenberg B: Platinum complexes for the treatment of cancer: why the research goes on. In: Cisplatin: Chemistry and Biochemistry of a Leading Anticancer Drug. Lippert B (ed). Verlag Helvetica Chemica Acta, Zurich, pp 3-12, 1999.
11. Hayes D M, Critkovic E, Golbey R B, et al: High dose cis-platinum diammine dichloride: amelioration of renal toxicity by mannitol diuresis. Cancer 39: 1372-1381, 1977.
12. Sorenson C M and Eastman A: Mechanism of cis-diamminedichlorplatinum (II)-induced cytotoxicity: role of G2 arrest and DNA double-strand breaks. Cancer Res 48: 4484-4488, 1988.
13. Gandara D R, Nahhas N A, Adelson M D, et al: Randomized placebo-controlled multicenter evaluation of diethyldithiocarbamate for chemoprotection against cisplatinum-induced toxicities. J Clin Oncol 13: 490-496, 1995.
14. Arany I and Safirstein R L: Cisplatin nephrotoxicity. Semin Nephrol 23: 460-464, 2003.
15. Tognoni A, Pensa F, Vaira F, et al: A dose finding study of carboplatin and gemcitabine in advanced non-small-cell lung cancer. J Chemother 14: 296-300, 2002.
16. Johnson B E: Integration of new agents into the treatment of advanced non-small-cell lung cancer. In: American Society of Clinical Oncology Educational Book. Perry M C, Govindan R (eds). Lipincott Williams and Wilkins, Baltimore, pp 345-356, 2000.
17. Stathopoulos G P, Veslemes M, Georgatou N, et al: Paclitaxel and vinorelbine combination in advanced inoperable adenocarcinoma of the lung: a phase II study. Anticancer Res 23: 3479-3484, 2003.
18. Stathopoulos G P, Dimitroulis J, Antoniou D, et al: Front-line paclitaxel and irinotecan combination chemotherapy in advanced non-small-cell lung cancer: a phase I-II trial. Br J Cancer 93: 1106-1111, 2005.
19. Fosella F V, DeVore R, Kerr R N, et al: Randomized phase III trial of docetaxel versus vinorelbine or ifosfamide in patients with advanced non-small-cell lung cancer previously treated with platinum-containing chemotherapy regimens. J Clin Oncol 18: 2354-2362, 2000.
20. Stathopoulos G P, Veslemes M, Georgatou N, et al: Front-line paclitaxel-vinorelbine versus paclitaxel-carboplatin in patients with advanced non-small-cell lung cancer: a randomized phase III trial. Ann Oncol 15: 1048-1055, 2004.
21. Comella P, Fresci G, Panza N, et al: Randomized trial comparing cisplatin, gemcitabine and vinorelbine with either cisplatin and gemcitabine or cisplatin and vinorelbine in advanced non-small cell lung cancer: interim analysis of a phase III trial of the Southern Italy Cooperative Oncology Group. J Clin Oncol 18: 1451-1457, 2000.
22. Boulikas T, Stathopoulos G P, Volakakis N and Vougiouka M: Systemic lipoplatin infusion results in preferential tumor uptake in human studies. Anticancer Res 25: 3031-3039, 2005.
23. Stathopoulos G P, Rigatos S K and Stathopoulos J: Liposomal cisplatin dose escalation for determining the maximum tolerated dose and dose-limiting toxicity: a phase I study. Anticancer Res 30: 1317-1321, 2010.
24. Stathopoulos G P, Boulikas T, Vougiouka M, et al: Liposomal cisplatin combined with gemcitabine in pretreated advanced pancreatic cancer patients: a phase I-II study. Oncol Rep 15: 1201-1204, 2005.
25. Koukourakis M, Giatromanolaki A, Pitakoudis M, et al: Concurrent liposomal cisplatin (lipoplatin), 5-fluorouracil and radiotherapy for the treatment of locally advanced gastric cancer: a phase I-II study. Int J Radiol Oncol Biol Phys 78: 150-155, 2009.
26. Karpathiou G, Argiana E, Koutsopoulos A and Froudarakis M E: Response of a patient with pleural and peritoneal mesothelioma after second-line chemotherapy with lipoplatin and gemcitabine. Oncology 73: 426-429, 2008.
27. Farhat F S, Ibrahim K, Kattan J, et al: Preliminary results of Phase II study of liposomal cisplatin-vinorelbine combination as first-line treatment in HER2/neu negative metastatic breast cancer (MBC). Proceedings of the ASCO Annual Meeting, abs. 31351, 2009.
28. Mylonakis M, Athanasiou A, Ziras N, et al: Phase II study of liposomal cisplatin (lipoplatin) plus gemcitabine versus cisplatin plus gemcitabine as first line treatment in inoperable (stage IIIB/IV) non-small-cell lung cancer. Lung Cancer 68: 240-247, 2010.
29. Stathopoulos G P, Boulikas T, Vougiouka M, et al: Pharmacokinetics and adverse reactions of a new liposomal cisplatin (lipoplatin): phase I study. Oncol Rep 13: 589-595, 2005.
30. Miller A B, Hoogstraten B and Stagnet M: Reporting results of cancer treatment. Cancer 47: 207-214, 1981.
31. Therasse P, Arbuck S G, Eisenhower E A, et al: New guidelines to evaluate the response to treatment in solid tumors. European Organization for Research and Treatment of Cancer, National Cancer Institute of the United States, National Cancer Institute of Canada. J Natl Cancer Inst 92: 205-216, 2000.
32. Stathopoulos G P, Rigatos S, Stathopoulos J and Batzios S: Liposomal cisplatin in cancer patients with renal failure. Proceedings of the ASCO Annual Meeting. abs. 7072, 2011.

1. A method for inhibiting the growth of a solid tumor or treating cancer in a patient comprising administering to the patient an effective amount of Lipoplatin monotherapy in a first dose and an effective amount of Lipoplatin monotherapy second dose, thereby inhibiting the growth of the solid tumor or treating cancer.

2. The method of claim 1, wherein the first dose is administered on day 1 and the second dose is administered between 12 to 36 hours after completion of the first dose.

3. The method of claim 1, wherein the first dose and the second dose are repeated two or more times, at intervals comprising 4 to 35 days there between.

4. The method claim 1, wherein the first dose comprises from about 100 mg/m² to 300 mg/m² Lipoplatin and the second dose comprises from about 100 mg/m² to 300 mg/m² Lipoplatin.

5. The method of claim 1, wherein the first dose comprise from about 150 mg/m² to 250 mg/m² Lipoplatin and the second dose are comprise from about 150 mg/m² to 250 mg/m² Lipoplatin.

6. The method of claim 1, wherein the solid tumor or cancer comprises metastatic or non-metastatic lung cancer, non-small cell lung cancer (NSCLC), breast cancer, Triple-negative breast cancer, gastric cancer, head and neck cancer, colon cancer, colorectal cancer, rectal cancer, pancreatic cancer, mesothelioma, brain cancer, (glioblastoma multiform or metastases) or ovarian cancer.

7. The method of claim 1, wherein the method comprises first line, second line or third line therapy.

8. The method of claim 1, wherein the patient has previously been treated with radiotherapy or oxaliplatin.

9. The method of claim 1, wherein the first dose and/or second dose is administered intravenously or by inhalation therapy.

10. The method of claim 6, wherein Lipoplatin is administered intravenously.

11. The method of claim 1, wherein the method is repeated with an interval of about 21 days to 35 days there between.

12. The method claim 1, wherein the method is repeated with an interval of about 26 days to 30 days there between.

13. The method of claim 1, further comprising administering an effective amount of a second chemotherapeutic agent.

14. The method of claim 13, wherein the second therapeutic agent is one or more of oxaliplatin, paclitaxel, vinorelbine or gemcitabine.

15. A method for inhibiting the growth of a solid tumor or treating cancer in a patient comprising administering a first dose of Lipoplatin monotherapy, wherein the first dose comprises about 200 mg/m² and a second dose of about 200 mg/m² of Lipoplatin monotherapy about 24 hours after administration of the first dose, thereby inhibiting the growth of the tumor or treating the patient.

16. The method of claim 15, wherein the cancer is lung cancer.

17. The method of claim 15, wherein the first dose and/or second dose is administered intravenously to the patient in a formulation comprising about 2 liters of a 5% Dextrose solution or saline.

18. The method of claim 15, further comprising one or more treatment cycles comprising repeating the first dose and the second dose is about every 14 days, after administration of the first dose.

19. The method of claim 18, wherein the one or more treatment cycles comprises at least 6 cycles of administration of the first dose and the second dose.

20. A method for inhibiting the growth of a solid tumor or treating cancer in a patient comprising administering a first dose of Lipoplatin monotherapy, wherein the first dose comprises about 200 mg/m2 and a second dose of about 200 mg/m2 of Lipoplatin monotherapy about 4 weeks after administration of the first dose, thereby inhibiting the growth of the tumor or treating the patient.

21. The method of claim 20, wherein the first dose and/or second dose is administered intravenously to the patient in a formulation comprising about 2 liters of a 5% Dextrose solution or saline.

22. The method of claim 20, further comprising one or more treatment cycles comprising repeating the first dose and the second dose as a maintenance therapy for the patient for life or until disease progression of the cancer or solid tumor.

23. A method for inhibiting the growth of a brain tumor or treating a brain tumor in a subject, comprising intra-arterial administration of an effective amount of Lipoplatin to the subject, thereby inhibiting the growth of the brain tumor or treating the brain tumor.

24. The method of claim 23, wherein the brain tumor is a glioblastoma multiform tumor or a tumor that has metastasized to the brain from a primary tumors outside the brain.

25. The method of claim 23 or 24, further comprising administering to the subject an effective amount of a one or more of a drug that enhances penetration and transport of Lipoplatin across the blood-brain-barrier (BBB), low dose radiation or oncothermia.

26. The method of claim 25, wherein the drug that enhances penetration and transport of Lipoplatin is temozolomide.

27. The method of claim 25, wherein the low dose radiation comprises one or more of an x-ray or a gamma knife.

28. A pharmaceutical Lipoplatin composition comprising an effective amount of Lipoplatin to provide a dose of from about 100 mg/m2 to about 300 mg/m2 to a subject in a pharmaceutically acceptable carrier.

29. The pharmaceutical composition of claim 28, further comprising an effective amount of a drug that enhances transport of the Lipoplatin across the blood brain barrier.

30. A kit comprising the composition of claim 28.
